(19) United States
(12) Patent Application Publication
(10) Pub. No.: US 2025/0205047 A1
(43) Pub. Date: Jun. 26, 2025

(54) Variable Wall Thickness Balloon for Crimp Profile Reduction and Balloon Shapes for Valve Inflation Stability

(71) Applicant: St. Jude Medical, Cardiology Division, Inc., St. Paul, MN (US)

(72) Inventors: Tyler Govek, St. Louis Park, MN (US); Hans Rieckmann, Minneapolis, MN (US); Neil Theisen, Champlin, MN (US)

(73) Assignee: St. Jude Medical, Cardiology Division, Inc., St. Paul, MN (US)

(21) Appl. No.: 18/935,810

(22) Filed: Nov. 4, 2024

Related U.S. Application Data
(60) Provisional application No. 63/614,744, filed on Dec. 26, 2023.

(51) Int. Cl. A61F 2/24; A61M 25/10

(52) U.S. Cl. CPC A61F 2/2433; A61F 2/2418; A61F 2230/0069; A61M 2025/1059

(57) ABSTRACT

According to an example of the disclosure, a system for delivering a prosthetic heart valve includes a handle, a delivery catheter extending from the handle, and a balloon on a distal portion of the catheter, the balloon having a proximal portion, a distal portion, and a central portion positioned between the proximal portion and the distal portion, the central portion configured to receive the prosthetic heart valve in a crimped condition thereon. The proximal portion may have a proximal wall thickness, the distal portion may have a distal wall thickness, and the central portion may have a central wall thickness, the central wall thickness being smaller than the proximal wall thickness and smaller than the distal wall thickness.

### CROSS-REFERENCE TO RELATED APPLICATIONS

This application claims priority to the filing date of U.S. Provisional Patent Application No. 63/614,744, filed Dec. 26, 2023, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

According to an example of the disclosure, a system for delivering a prosthetic heart valve includes a handle, a delivery catheter extending from the handle, and a balloon on a distal portion of the catheter, the balloon having a proximal portion, a distal portion, and a central portion positioned between the proximal portion and the distal portion, the central portion configured to receive the prosthetic heart valve in a crimped condition thereon. The proximal portion may have a proximal wall thickness, the distal portion may have a distal wall thickness, and the central portion may have a central wall thickness, the central wall thickness being smaller than the proximal wall thickness and smaller than the distal wall thickness. The proximal wall thickness may be about equal to the distal wall thickness. The proximal portion of the balloon, the distal portion of the balloon, and the central portion of the balloon may be formed integrally with each other. The proximal portion of the balloon and the distal portion of the balloon may each have a compliance that is greater than a compliance of the central portion of the balloon. The central wall thickness may be between about 25% and about 75% of the proximal wall thickness. The central wall thickness may be about 50% of the proximal wall thickness. The system may include the prosthetic heart valve, and the prosthetic heart valve may be a balloon-expandable prosthetic heart valve, the system having a delivery configuration in which the prosthetic heart valve is crimped so that it overlies the central portion of the balloon but does not overlie the proximal portion of the balloon or the distal portion of the balloon. In the delivery configuration of the system, the proximal portion of the balloon and the distal portion of the balloon may each have an outer diameter that is greater or equal to an outer diameter of the crimped prosthetic heart valve. The balloon may have a deflated delivery condition and an inflated deployment condition, an outer diameter of the distal portion of the balloon being greater than an outer diameter of the central portion of the balloon in the inflated deployment condition. An outer diameter of the proximal portion of the balloon may be greater than the outer diameter of the central portion of the balloon in the inflated deployment condition.

According to an example of the disclosure, a method of manufacturing a system for delivering a prosthetic heart valve may include extruding a polymer into a tube, stretching or necking down the extruded tube to form a parison, positioning the parison within a mold, pressurizing the parison within the mold to form a balloon, and coupling the formed balloon to a distal end of a delivery catheter of a delivery device. The formed balloon may include a proximal portion having a proximal wall thickness, a distal portion having a distal wall thickness, and a central portion having positioned between the proximal portion and the distal portion and having a central wall thickness, the central wall thickness being smaller than the proximal wall thickness and smaller than the distal wall thickness. The method may include stretching the parison, prior to positioning the parison within the mold, to form the central wall thickness to be smaller than the proximal wall thickness and the distal wall thickness. The method may include coating the parison or the formed balloon to increase the proximal wall thickness and the distal wall thickness relative to the central wall thickness. Coating the parison may include dip coating the parison. The proximal portion and the distal portion of the balloon may each have a compliance that is greater than a compliance the central portion of the balloon. The proximal portion and the distal portion of the balloon may each be formed as a coextrusion of two materials, and the central portion of the balloon may be formed as an extrusion of one material. The method may include crimping the prosthetic heart valve over the central portion of the balloon after coupling the balloon to the distal end of the delivery catheter of the delivery device so that the proximal portion of the balloon and the distal portion of the balloon are not covered by the crimped prosthetic heart valve. After crimping the prosthetic heart valve over the central portion of the balloon, an outer diameter of the crimped prosthetic heart valve may be equal to or smaller than an outer diameter of the distal portion of the balloon. The mold may have a proximal portion with a proximal interior diameter, a distal portion with a distal interior diameter, and a central portion with a central interior diameter that is smaller than the proximal interior diameter and the distal interior diameter. The mold may be formed of two pieces, and prior to pressurizing the parison, the two pieces of the mold may be coupled together.

According to another example of the disclosure, a system for delivering a prosthetic heart valve may include a handle, a delivery catheter extending from the handle, and a balloon on a distal portion of the catheter, the balloon having a proximal portion, a distal portion, and a central portion positioned between the proximal portion and the distal portion, the central portion configured to receive the prosthetic heart valve in a crimped condition thereon. When the balloon is in an inflated or blown state, the proximal portion may have a proximal outer diameter, the distal portion may have a distal outer diameter, and the central portion may have a central outer diameter that is smaller than the proximal outer diameter and smaller than the distal outer diameter. The proximal portion of the balloon, the distal portion of the balloon, and the central portion of the balloon may be formed integrally with each other. The system may also include the prosthetic heart valve, which may be a balloon-expandable prosthetic heart valve, and the system may have a delivery configuration in which the prosthetic heart valve is crimped so that it overlies the central portion of the balloon but does not overlie the proximal portion of the balloon or the distal portion of the balloon. In an inflated condition of the balloon, a transition between the proximal portion of the balloon and the central portion of the balloon may form a proximal shoulder, and a transition between the distal portion of the balloon and the central portion of the balloon may form a distal shoulder. In the inflated condition of the balloon, an inflow end of the prosthetic heart valve may abut the distal shoulder and an outflow end of the prosthetic heart valve may abut the proximal shoulder. The proximal portion of the balloon may have a proximal wall thickness, the distal portion of the balloon may have a distal wall thickness, and the central portion of the balloon may have a central wall thickness, the central wall thickness being smaller than the proximal wall thickness and smaller than the distal wall thickness. The proximal wall thickness may be about equal to the distal wall thickness. The proximal portion of the balloon and the distal portion of the balloon may each have a compliance that is greater than a compliance of the central portion of the balloon. The central wall thickness may be between about 25% and about 75% of the proximal wall thickness. The central wall thickness may be about 50% of the proximal wall thickness.

According to another example of the disclosure, a method of manufacturing a system for delivering a prosthetic heart valve may include extruding a polymer into a tube, stretching or necking down the extruded tube to form a parison, positioning the parison within a mold, pressurizing the parison within the mold to form a balloon, and coupling the formed balloon to a distal end of a delivery catheter of a delivery device. The mold may have a proximal portion with a proximal interior diameter, a distal portion with a distal interior diameter, and a central portion with a central interior diameter that is smaller than the proximal interior diameter and the distal interior diameter. The mold may include a proximal mold removably coupled to a distal mold, and prior to pressurizing the parison, the proximal mold may be coupled to the distal mold. The proximal mold may include a proximal opening and the distal mold may include a distal opening, the proximal opening and the distal opening being sized and shaped to receive the extruded tube therethrough after the extruded tube is stretched or necked down. The proximal mold may include a distal connector, and the distal mold may include a proximal connector, the proximal connector configured to couple to the distal connector to couple the proximal mold to the distal mold. The proximal connector may be formed of threading and the distal connector may be formed of threading. The central portion of the mold may be formed partly by the proximal mold and partly by the distal mold. The formed balloon may include a proximal portion having a proximal wall thickness, a distal portion having a distal wall thickness, and a central portion having positioned between the proximal portion and the distal portion and having a central wall thickness, the central wall thickness being smaller than the proximal wall thickness and smaller than the distal wall thickness. The method may include stretching the parison, prior to positioning the parison within the mold, to form the central wall thickness to be smaller than the proximal wall thickness and the distal wall thickness. The method may include coating the parison or the formed balloon to increase the proximal wall thickness and the distal wall thickness relative to the central wall thickness. Coating the parison, may include dip coating the parison. The mold may include a top mold removably coupled to a bottom mold, and prior to pressurizing the parison, the top mold may be coupled to the bottom mold. The top mold and the bottom mold may each define a portion of a proximal opening and a portion of a distal opening, such that when the top mold is coupled to the bottom mold, the proximal opening and the distal opening are formed and are sized and shaped to receive the extruded tube therethrough after the extruded tube is stretched or necked down.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12A illustrates an example of a balloon of a balloon catheter, with the balloon being in a blown or inflated state and having a variable wall thickness.

FIG. 12B is a flow chart showing example steps of an exemplary method of forming a balloon for a balloon catheter having a variable wall thickness.

FIG. 13A illustrates another example of a balloon of a balloon catheter, with the balloon being in a blown or inflated state and having a variable wall thickness.

FIG. 13B illustrates the balloon of FIG. 13A in a collapsed state with a prosthetic heart valve crimped thereon.

FIG. 13C illustrates the assembly of FIG. 13B with the balloon in an inflated state and the prosthetic heart valve in an expanded condition.

FIG. 14A is a highly schematic example of two portions of a blow mold in an uncoupled condition.

FIG. 14B is a highly schematic example of the two portions of the blow mold of FIG. 14A assembled with the parison of an extruded tube positioned therein.

FIG. 14C is a highly schematic example of the assembled blow mold of FIG. 14B after the parison has bene pressurized and the balloon is in the blown state.

FIG. 14D is a highly schematic example of the blow mold of FIG. 14C being disassembled after the balloon has been formed.

FIG. 15A is highly schematic end view of another example of two portions of a blow mold in an uncoupled condition.

FIG. 15B is a highly schematic side view of the two portions of the blow mold of FIG. 15A assembled together.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

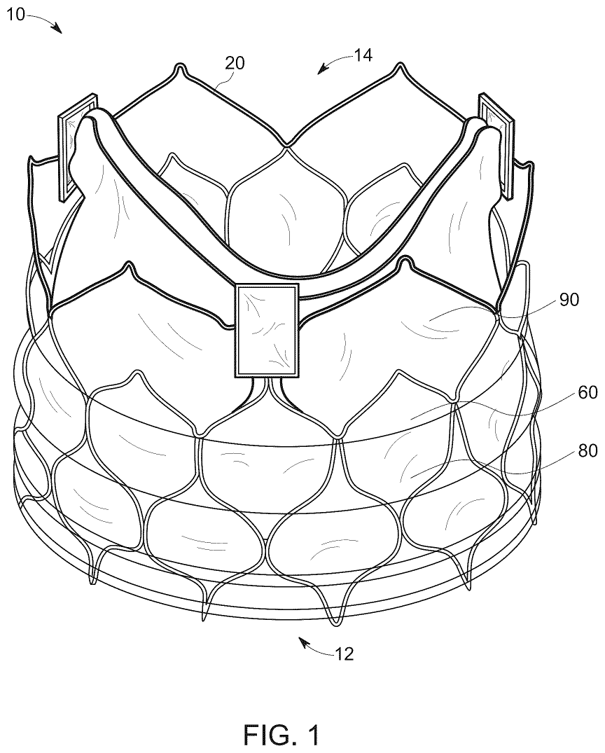

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

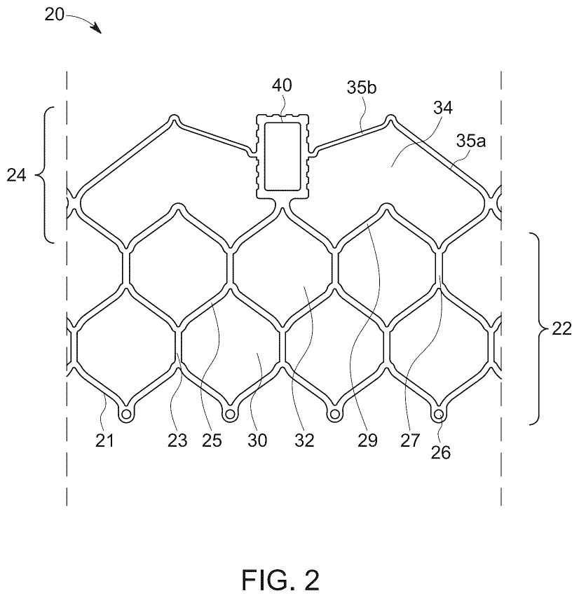

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g., fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g., struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

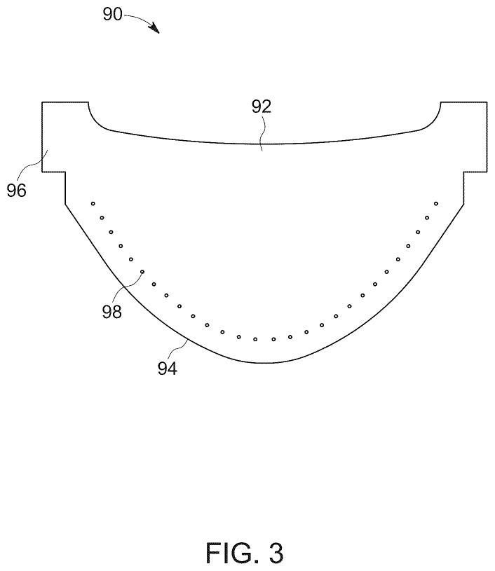

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g., via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

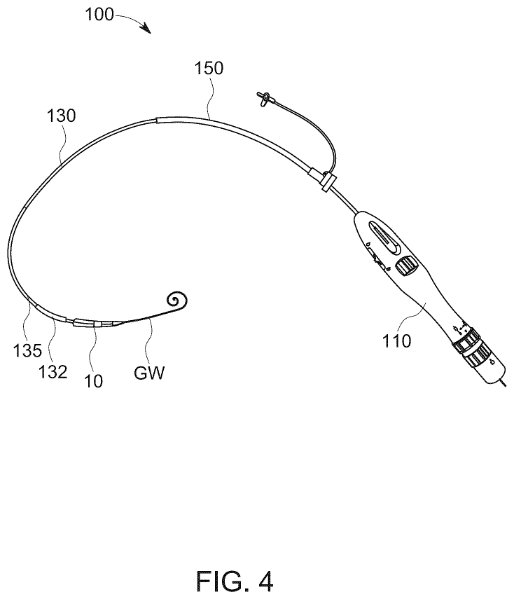

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

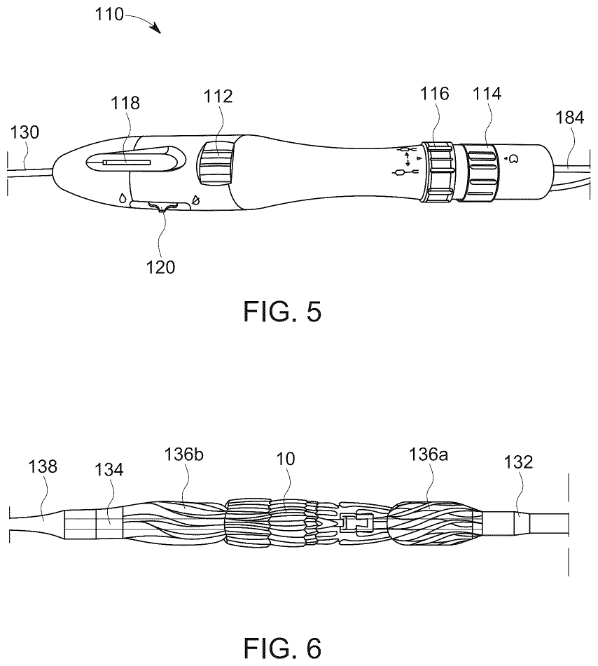

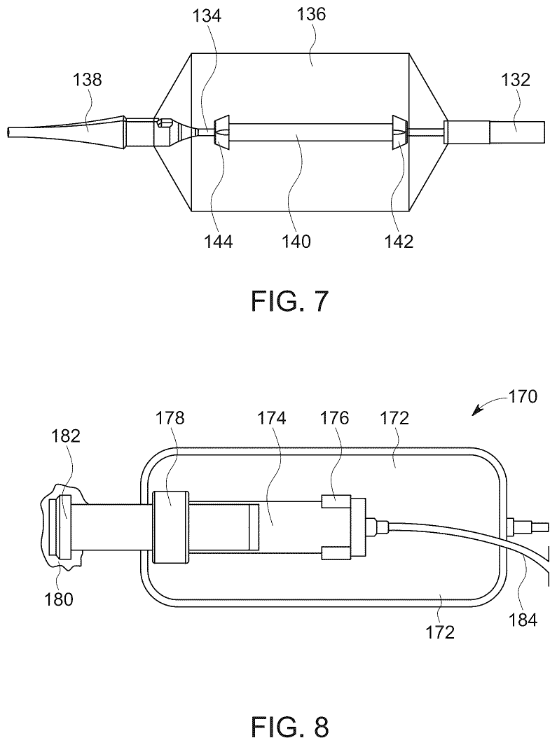

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g., the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to the inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

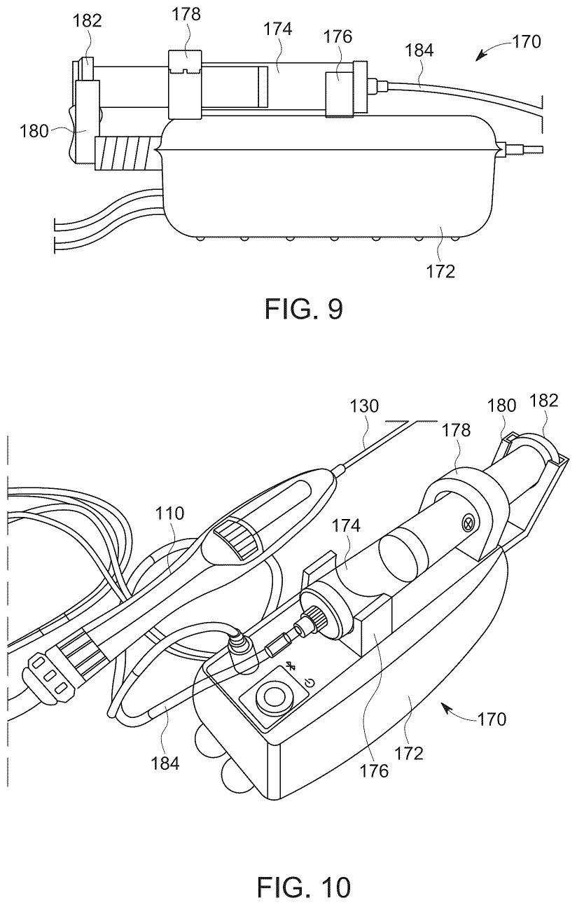

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g., from AC mains or another component with which the cable is coupled) and/or transmission of data, information, control commands, etc. For example, one cable may couple the housing 172 to handle 110 so that controls on the handle 110 (e.g., balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

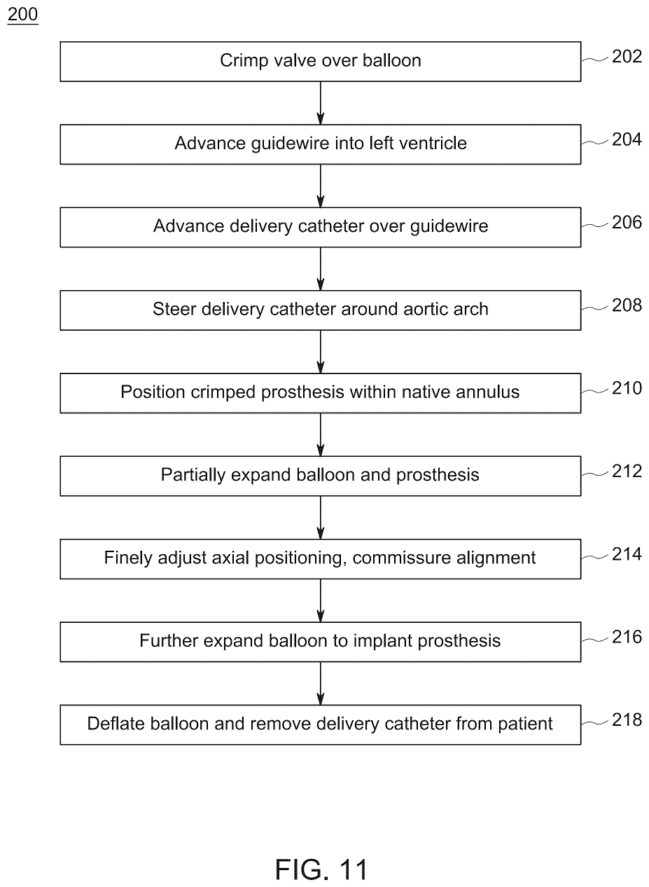

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g., by actuating axial alignment actuator 116) and/or the rotational orientation of the prosthetic heart valve 10 relative to the native aortic valve may be finely adjust (e.g., by actuating commissure alignment actuator 114). When the desired axial alignment is achieve and the desired rotational alignment (e.g., rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel. In one example, the delivery catheter 130 may be guided to the right atrium and/or right ventricle for a tricuspid valve or pulmonary valve procedure. In another example, the delivery catheter 130 may be guided to the left atrium and/or left ventricle for a mitral valve procedure.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

For many transcatheter prosthetic heart valve replacement procedures, the size of the system that needs to pass through a patient's vasculature is an important parameter. For example, as the outer diameter of the system that needs to pass through the patient's vasculature increases, risks of damaging the patient's tissue may increase, while other aspects of the procedure, such as accurate steering, may become more difficult as the size increases. As used herein, the term “crimp profile” refers to the outer diameter of a prosthetic heart valve (e.g., valve 10) while it is crimped in a collapsed condition over a balloon (e.g., balloon 136) of delivery device (e.g., delivery system 100) in a condition ready for delivery. In some examples, components that may contribute to the crimp profile include the prosthetic heart valve itself (which may include components including a frame, prosthetic leaflets, and one or more skirts or cuffs), the balloon, and any interior catheter components such as an inner catheter or guidewire catheter. There are multiple approaches to reduce the crimp profile of a prosthetic heart valve system. Many such approaches focus on modifying the prosthetic heart valve itself. However, as described in greater detail below, impactful gains in crimp profile reduction may be achieved via modification of the balloon.

One potentially important feature of a balloon (e.g., balloon 136) of a delivery device for a balloon-expandable prosthetic heart valve is the ability to withstand bursting at operationally relevant balloon pressures. Another potentially important feature of the balloon is fatigue, which may refer to the ability of the balloon to undergo multiple cycles of inflation/deflation at operationally relevant balloon pressures. Burst and fatigue testing are frequently performed on the “naked” balloon—i.e., without the prosthetic heart valve crimped over the balloon. In one example of modifying the balloon to reduce crimp profile, the wall thickness of the balloon may be reduced, either over the entire surface of the balloon or at specific locations of the balloon. In this example, removing material results in less bulk of the balloon, and therefore, a potential in reduced crimp profile. Thinning the wall of the balloon, either over the entire surface of the balloon or at selected locations on the balloon, will typically negatively affect burst and/or fatigue testing of a naked balloon. However, when a prosthetic heart valve is crimped over the balloon, the results of the burst and/or fatigue testing improve compared to when the same balloon is tested as a naked balloon.

Another potentially important feature of a balloon (e.g., balloon 136) of a delivery device for a balloon-expandable prosthetic heart valve is the ability to maintain a stable and/or predictable position of the prosthetic heart valve on the balloon as the balloon inflates. The example of balloon 136 shown and described in connection with FIG. 6 includes a proximal pillowed portion 136a and a distal pillowed portion 136b that may act as shoulders to keep the prosthetic heart valve 10 in a stable position between the two pillowed portions during delivery. However, as balloon 136 expands, it may in some examples expand so that the pillowed portions are no longer structurally distinct from the rest of the balloon 136. For example, as shown in the example of FIG. 7, when the balloon 136 is expanded, the pillowed portions may effectively disappear.

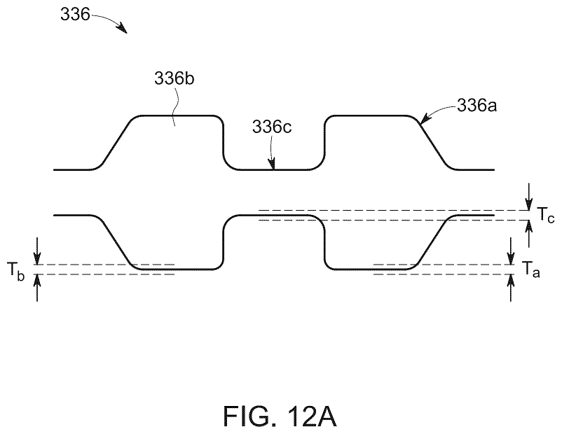

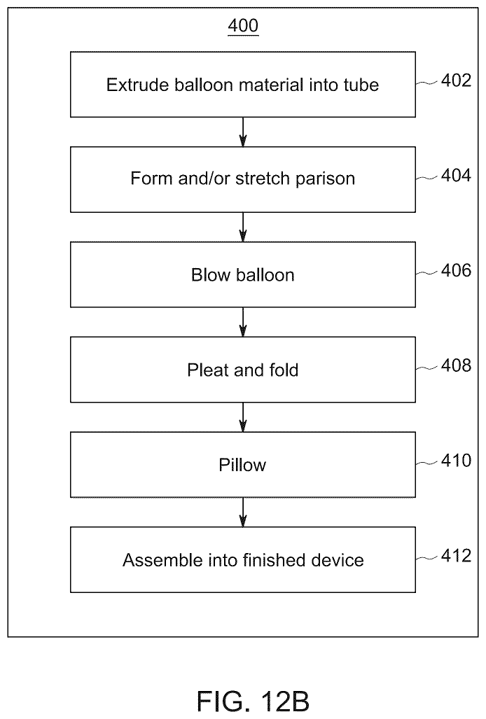

Referring now in addition to FIG. 12A, FIG. 12A illustrates a balloon 336 that may be used with a delivery system similar or identical to delivery system 100. For example, balloon 136 may be replaced with balloon 336 without needing any additional modifications to the delivery system 100. However, in other examples, balloon 336 may be used with a balloon catheter system different than delivery system 100. In the example of FIG. 12A, balloon 336 is shown in a blown state (which may also correspond to the inflated state), but before additional processing as described in greater detail below. Balloon 336 in some examples includes a proximal pillowed portion 336a and a distal pillowed portion 336b that each have an outer diameter that is larger than an outer diameter of a central balloon portion 336c which may also be referred to as the balloon crimp zone, which may or may not be positioned at a mathematical center of the balloon 336. In some embodiments, the outer diameters of the proximal pillowed portion 336a and the distal pillowed portion 336b are the same. In other examples, the outer diameter of the proximal pillowed portion 336a may be larger or smaller than the outer diameter of the distal pillowed portion 336b. It should be understood that the outer diameters of the balloon portions 336a-336c shown in FIG. 12A are not necessarily to scale, and in some examples the size difference between the central balloon portion 336c and the proximal and distal pillowed portions 336a, 336b is exaggerated for purposes of illustration. Further, in some embodiments, the outer diameters of the three balloon portions 336a, 336b, 336c may be substantially the same when the balloon 336 is inflated. In some examples the balloon 336 is formed as a single integral member. In some examples of balloon 336 (as well as balloon 136 and balloon 536), the crimp zone 336c may have a length that is about equal to an axial length of the prosthetic heart valve (e.g., valve 10) when the prosthetic heart valve is crimped over the crimp zone 336c. In some examples of balloon 336 (as well as balloon 136 and balloon 536), the proximal pillowed portion 336a (if included) and/or the distal pillowed portion 336b (if included) are each positioned immediately adjacent to the crimp zone 336c on opposite sides of the crimp zone 336c.

Balloon 336 in some examples may be formed with sections having two or more wall thicknesses. For example, the proximal pillowed portion 336a may be formed to have a wall thickness Tₐ, the distal pillowed portion 336b may be formed to have a wall thickness T_{b}, and the central portion or crimp zone 336c may be formed to have a wall thickness T_{c}. In the illustrated example, the wall thickness T_{c} is smaller (i.e., thinner) than both wall thicknesses Tₐ and T_{b}. In some examples, wall thicknesses Tₐ and T_{b} may be substantially the same, although in other examples wall thickness Tₐ may be smaller than (i.e., thinner) or larger than (i.e., thicker) than wall thickness T_{b}. In some examples, the wall thickness T_{c} of the crimp zone 336c may be between about 25% to about 75%, including about 50%, the wall thickness Tₐ of the proximal pillowed portion 336a and/or the wall thickness T_{b} of the distal pillowed portion 336b. In one particular example, the wall thickness T_{c} of the crimp zone 336c, measured as double wall thickness (“DWT”) may be between about 0.002 inches (about 0.051 mm) and about. 004 inches (about 0.101 mm), including about 0.003 inches (about 0.076 mm) and the wall thicknesses Tₐ, T_{b} may each be between about 0.005 inches (about 0.127 mm) and about 0.007 inches (about 0.178 mm), including about 0.006 inches (about 0.152 mm) DWT. For the particular examples of measurements provided immediately above, the single wall thickness would be about half the double wall thickness. The particular examples above, which should not be considered limiting but rather exemplary, may include thicknesses that are greater or smaller than the listed values by about 10%. With the configuration described in connection with FIG. 12A, the thinner crimp zone 336c in some examples results in a smaller crimp profile of the crimp zone 336c compared to an otherwise identical balloon in which the wall thickness T_{c} is larger (i.e., thicker) so that it is about equal to wall thicknesses Tₐ and T_{b}. In other words, in examples in which the crimp zone 336c is thinner, there is less material that needs to be collapsed and/or folded and/or twisted when the prosthetic heart valve 10 is crimped over the crimp zone 336c (similar to the configuration shown in connection with FIG. 6). Thus, when the prosthetic heart valve (e.g., prosthetic heart valve 10) is crimped on balloon 336, the outer diameter of the crimped prosthetic heart valve may be smaller than if the prosthetic heart valve were crimped on a different balloon with a uniform balloon wall thickness. In some examples, one or more (including all) of the wall thicknesses Tₐ, T_{b}, T_{c} of the corresponding sections of the balloon 336 may have a substantially uniform thickness (e.g., less than 10% deviation or less than 5% deviation) within the section. In some examples, or more (including all) of the wall thicknesses Tₐ, T_{b}, T_{c} of the corresponding sections of the balloon 336 may have a varying thickness within the section, but these examples the average thickness of each section includes relationships described above. In other words, in these examples with varying wall thickness within a section, the average wall thickness T_{c} is smaller (i.e., thinner) than both average wall thickness Tₐ and average wall thickness T_{b}. In these examples with varying wall thickness within a section, in some examples average wall thicknesses Tₐ and T_{b} may be substantially the same, although in other examples average wall thickness Tₐ may be smaller than (i.e., thinner) or larger than (i.e., thicker) than average wall thickness T_{b}.

Now referring in addition to FIG. 12B, FIG. 12B illustrates exemplary steps of one exemplary method 400 for manufacturing balloon 336. In an example of a first step 402 of method 400, the material that will form the balloon 336 may be extruded into a tube-like shape. In some examples, the material being extruded to form the balloon 336 may be a polymer. In some examples, the material may be a polyether block amide such as that offered under the tradename Pebax®. In some examples, the material being extruded to form the balloon 336 may be a nylon material. In other examples, the material being extruded to form the balloon 336 may be a polyester, a polyurethane, or a silicone. In some examples, one or more of these materials may be combined, for example, into a coextrusion. It should be understood that these materials are exemplary only. In an example of a second step 404, after the material has been extruded into a tube, the tube may be stretched or necked down to form a parison. In an example of a third step 406, after the parison has been formed via stretching or necking down the extruded tube, the balloon may be formed via blow molding. In some examples of third step 406, the parison may be placed inside of a mold having an interior shape that is similar or identical to the desired final shape of the balloon 336 after it is formed and inflated. The parison may be internally pressurized to expand the parison into contact with the interior surface of the mold so that the desired balloon shape is formed. In some examples, the third step 406 of blow molding may be completed by allowing the balloon 336 to cool to effectively “set” the shape of the balloon when inflated. In some examples, after blow molding, additional processing may be performed in which some or all areas of the balloon 336 may be pleated, folded, and/or twisted in a fourth step 408 to help minimize the profile of the balloon 336 when it is in a collapsed or deflated state. In some examples, a fifth step 410 may be performed to create the “pillowed” sections, for example a proximal pillowed section (e.g., 136a in FIG. 6 or 336a in FIG. 12A) and/or a distal pillowed section (e.g., 136b in FIG. 6 or 336b in FIG. 12A). In some examples, the fifth step 410 may be performed by applying heat to the relevant areas of the balloon 336 while the proximal and/or distal sections have the “pillowed” shape, although other suitable methods for forming the pillows are described in U.S. patent application Ser. No. 18/498,556, filed Oct. 31, 2023, the disclosure of which is hereby incorporated by reference herein. In an example of a sixth step 412, the balloon 336 may be assembled to the rest of the delivery system (e.g., to delivery system 100 via connection to the inner catheter 134 and/or nosecone 138) and/or to the prosthetic heart valve (e.g., by crimping prosthetic heart valve 10 over the crimp zone 336c.

Referring still to FIG. 12B, in one example of first step 402, a substantially uniform material is extruded into the tube, for example a single Pebax or nylon material, or a substantially uniform combination of two or more materials that are coextruded into the tube. In order to create a crimp zone 336c with reduced wall thickness, in one example, stretching (or differential stretching) of the parison may be performed in second step 404 to fine tune the wall thicknesses as desired. In one particular example of this stretching (or differential stretching), the parison may be clamped on two sides and the clamped sections may be pulled apart at relatively high speeds and/or to a relatively large distance to create a relatively thin wall which will eventually form (in step 406 for example) the crimp zone 336c. Then, in a second step, the parison may be clamped at locations a spaced distance from each end of the relatively thin-walled section and stretched apart at a relatively low speed and or to a relatively small distance to create relatively thin walls which will eventually form (in step 406 for example) the proximal pillowed portion 336a and the distal pillowed portion 336b. In some examples, the first and second steps may be performed simultaneously, for example with three or four clamped sections being stretched simultaneously, but at different rates and/or to different distances. However, it should be understood that other specific methods of stretching (or differential stretching) may be used to achieve the different wall thicknesses if the different wall thicknesses are to be achieved via stretching. In this particular example, even though the smaller wall thickness T_{c} might otherwise reduce the burst strength of the balloon 336 at the crimp zone 336c, the support provided by the prosthetic heart valve 10 crimped over the crimp zone 336c may increase the effective burst strength of the balloon 336 at the crimp zone 336c in some examples.

Although the example immediately above addresses one or more ways to achieve a relatively small wall thickness T_{c} by differential stretching of a substantially uniform parison, in other examples different wall thicknesses Tₐ, T_{b}, T_{c} may be achieved by the use of coatings and/or coextrusions in selected areas of the balloon 336. For example, referring back to FIG. 12B, in one example of first step 402, a substantially uniform material is extruded into the tube, for example a single Pebax or nylon material, or a substantially uniform combination of two or more materials that are coextruded into the tube. In order to create a crimp zone 336c with reduced wall thickness, in one example, either prior to, simultaneously with, or after the second step 404 of forming the parison, the portions of the material that will be formed into the proximal pillow portion 336a and the distal pillow portion 336b may be coated, for example via dip coating, with a different and/or additional material. In some examples, if dip coating is performed, it may be performed on the extruded tube prior to forming the parison, on the parison prior to blowing, or on the balloon after blowing the parison into the balloon (including at any one of these stages of manufacture or at any two or all three stages of manufacture). By forming the proximal pillow portion 336a and the distal pillow portion 336b with an additional coating, in some examples the wall thicknesses Tₐ, T_{b}, may be increased relative to the wall thickness T_{c} of the crimp zone 336c and/or the different segments of the balloon 336 may be provided with different material properties. As an example, the crimp zone 336c may be formed with a relatively high durometer Pebax or nylon so that the crimp zone 336c is relatively rigid, while the proximal pillow portion 336a and distal pillow portion 336b may be dip coated with a relatively low durometer Pebax or nylon. With this type of configuration, the crimp zone 336c may be formed of a relatively thin-walled rigid material, while the proximal pillow portion 336a and the distal pillow portion 336b may be formed of a relatively thick-walled compliant material. In addition to helping to achieve a smaller profile balloon 336, this configuration may in some examples result in better stability of the prosthetic heart valve 10 during deployment. For example, if the proximal pillow portion 336a and the distal pillow portion 336b are more compliant than the crimp zone 336c, during inflation of the balloon 336, the proximal pillow portion 336a and distal pillow portion 336b may tend to inflate earlier or faster than the crimp zone 336c, maintaining the shoulders of the proximal pillow portion 336a and the distal pillow portion 336b during most or all of the inflation of the balloon 336. In some examples, by maintaining the shoulders during most or all of the inflation, the prosthetic heart valve 10 will tend to maintain a constant axial position because it is “book-ended” by the shoulders during most or all of the inflation.

While the example immediately above describes coating (e.g., dip coating) the proximal pillow portion 336a and the distal pillow portion 336b, in some examples a similar result may be achieved by using a coextrusion selectively in the areas that will be formed into the proximal pillow portion 336a and the distal pillow portion 336b, while using a single extrusion in the areas that will be formed into the crimp zone 336c. For example, the area that will become the crimp zone 336c may be formed of a single extrusion of a relatively high durometer Pebax or nylon, while the areas that will become the proximal pillow portion 336a and the distal pillow portion 336b may be formed of a coextrusion of the relatively high durometer Pebax or nylon, with an additional layer of relatively low diameter Pebax or nylon. By forming the balloon 336 using method 400 with these selective areas of coextrusion, the resulting balloon 336 may in some examples have a relatively thin-walled rigid crimp zone 336c and relatively thick-walled compliant proximal and distal pillowed portions 336a, 336b.

While the example immediately above describes using coextrusion selectively in different areas of the balloon 336, in other examples, in step 402 a single extruded tube may be formed with areas formed of only a single material of relatively high durometer and other areas of only a single material of relatively low durometer. For example, a central section of the extruded tube may be formed of a relatively high durometer Pebax or nylon, with sections of the extruded tube on each side of the central section being formed of a relatively low durometer Pebax or nylon. This extruded tube may be formed into balloon 336 using method 400, which in some examples may include the parison stretching (and/or differential stretching) in the second step 404 described above. With this example, the balloon 336 may be formed to include a relatively thin-walled rigid crimp zone 336c and relatively thick-walled compliant proximal and distal pillowed portions 336a, 336b.

In other examples, either instead of or in addition to the extrusion and/or stretching steps described above, the desired wall thicknesses of the different balloon sections (or desired ranges of wall thickness of the different balloon sections) may be obtained via additive manufacturing (e.g., 3D printing). In other words, in some examples, the balloon may be formed using typical blow molding to achieve a uniform thickness among the different sections, and certain sections may be increased in thickness to the desired value via 3D printing. In other examples, the entire balloon may be constructed to have the desired thicknesses via 3D printing the balloon, without needing separate extrusion/stretching/blowing steps.

Referring again to FIG. 12A, balloon 336 is described in some examples as including a blown state in which the proximal and distal portions of the balloon form larger-diameter pillowed portions compared to a smaller-diameter crimp zone 336c. As noted above, these examples may assist in more stable positioning of the prosthetic heart valve 10 during inflation of the balloon 336. However, it should be understood that the crimp profile reduction achieved by having a thinner-walled crimp zone 336c does not necessarily require that the portions of the balloon 336 proximal and distal to the crimp zone 336c have a larger diameter in the blown state. In other words, in the blown state, the balloon 336 may have a shape generally similar to that shown in FIG. 7, while still having a thinner-walled crimp zone 336c (and thicker-walled balloon portions distal to and/or proximal to the crimp zone 336c) so that an overall reduction of crimp profile may be achieved.

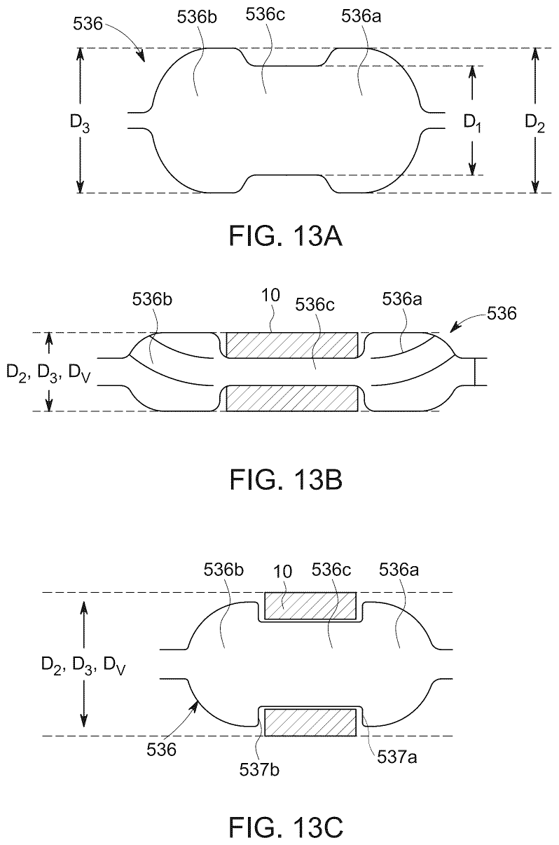

Referring now in addition to FIG. 13A, FIG. 13A illustrates a balloon 536 that may be used with a delivery system similar or identical to delivery system 100. In some examples, balloon 536 includes one or more features or characteristics of other balloons disclosed herein and may be used or manufactured in a similar manner. For example, balloon 136 and/or balloon 336 may be replaced with balloon 536 without needing any additional modifications to the delivery system 100. However, in other examples, balloon 536 may be used with a balloon catheter system different than delivery system 100. In the example of FIG. 13A, balloon 536 is shown in a blown state (which may also correspond to the inflated state), but before additional processing. Whereas the examples of balloon 336 described above generally focus on a thinned-wall balloon section to reduce crimp profile with an option of pillowed sections for axially maintaining the prosthetic heart valve (e.g., prosthetic heart valve 10) during deployment, the examples of balloon 536 described below generally focus on pillowed sections for axially maintaining the prosthetic heart valve (e.g., prosthetic heart valve 10) during deployment with an option of a thinned-wall balloon section to reduce crimp profile.

Balloon 536 in some examples includes a proximal pillowed portion 536a and a distal pillowed portion 536b that each have an outer diameter (D₂, D₃, respectively) that is larger than an outer diameter D₁ of a central balloon portion 536c which may also be referred to as the balloon crimp zone. In some embodiments, the outer diameters D₁, D₂, of the proximal pillowed portion 536a and the distal pillowed portion 536b are the same. In other examples, the outer diameter D₁ of the proximal pillowed portion 536a may be larger or smaller than the outer diameter D₂ of the distal pillowed portion 536b. It should be understood that the outer diameters D₁-D₃ of the balloon portions 536a-536c shown in FIG. 13A are not necessarily to scale. In some examples, the balloon 536 is formed as a single integral member.

Referring now in addition to FIG. 13B, FIG. 13B illustrates prosthetic heart valve 10 (although it should be understood that other examples of balloon expandable heart valves beyond prosthetic heart valve 10 may be suitable alternatives) crimped over the crimp zone 536c while the balloon 536 is in a deflated state. In the example of FIG. 13B, the proximal pillow portion 536a and distal pillow portion 536b each exist in the (mostly or entirely) deflated condition of the balloon 536, similar or identical to the condition shown in FIG. 6. As described in connection with FIG. 6, the existence of the pillowed portions 536a, 536b during delivery of prosthetic heart valve 10 may in some examples help maintain the axial position of the prosthetic heart valve 10 relative to the balloon 536 during delivery (e.g., by “book-ending” the valve), while also in some examples protecting the leading edge of the prosthetic heart valve 10 from contacting the vasculature and potentially dislodging the prosthetic heart valve 10. In the example of FIG. 13B, while the balloon 536 is mostly or entirely deflated and the prosthetic heart valve 10 is crimped thereon in a delivery condition, in some examples the outer diameters of the proximal pillowed portion 536a, the prosthetic heart valve 10, and the distal pillowed portion 536b (D₂, D_{V}, D₃ respectively) are all substantially equal. However, in other examples, the outer diameter D_{V} of the prosthetic heart valve 10 is smaller than the outer diameter D₃ of the distal pillowed portion 536b (and in some examples equal to or smaller than the outer diameter D₂ of the proximal pillowed portion 536a).

Referring now in addition to FIG. 13C, FIG. 13C illustrates the assembly of FIG. 13B with the balloon 536 in an inflated state and prosthetic heart valve 10 in an expanded condition. In some examples, during inflation of the balloon 536, including when the balloon 536 has inflated enough to deploy prosthetic heart valve 10, the outer diameters of the proximal pillowed portion 536a, the prosthetic heart valve 10, and the distal pillowed portion 536b (D₂, D_{V}, D₃ respectively) are all substantially equal, with the prosthetic heart valve 10 received within a recessed area defined by the crimp zone 536c. However, in other examples, the outer diameter D_{V} of the prosthetic heart valve 10 is smaller than the outer diameter D₃ of the distal pillowed portion 536b and the outer diameter D₂ of the proximal pillowed portion 536a. In these exemplary configurations, a proximal shoulder 537a is formed where the outflow end of the prosthetic heart valve 10 contacts the proximal pillowed portion 536a, and a distal shoulder 537b is formed where the inflow end of the prosthetic heart valve 10 contacts the distal pillowed portion 536b. These shoulders 537a, 537b may exist not only when the balloon 536 is mostly or entirely deflated and the prosthetic heart valve 10 is in the crimped condition for delivery, but also during most or all of the process of inflating the balloon 536 to deploy the prosthetic heart valve 10.

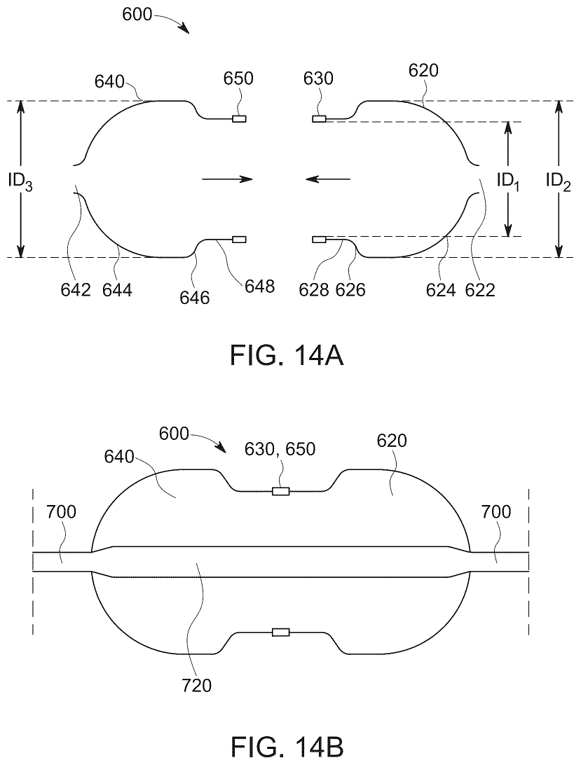

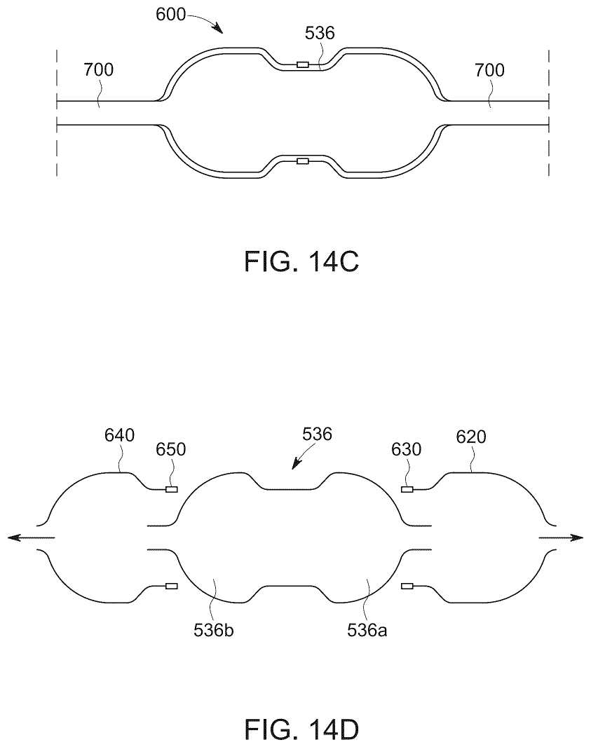

In some examples, balloon 536 may be manufactured with a method including one or more steps of method 400 described above, with or without one or more modifications. In some examples, the fourth step 408 of pleating and folding may be included when forming balloon 536, with FIG. 13B illustrating an example of such pleating and folding in the proximal pillowed portion 536a and distal pillowed portion 536b. In some examples, in the exemplary first step 402, and second step 404, the balloon material may be extruded into a tube using any of the examples described above in connection with balloon 336 (e.g., to form a relatively thin-walled crimp zone 536c), although in other examples the tube extrusion and parison may be formed in a more traditional manner without creating a thin-walled crimp zone 536c. However, when blow molding the balloon 536 in exemplary third step 406, a specialized mold may be used to create the recessed diameter D₁ of the crimp zone 536c when the balloon 536 is in the inflated state. Examples of particular structures and methods in connection with exemplary third step 406 are described in greater detail below in connection with FIGS. 14A-D, and it should be understood that these exemplary structures and methods may be used to form the balloon 336 as well if desired.

Referring now in addition to FIG. 14A, FIG. 14A is a highly schematic example of two portions of an example of a blow mold 600 in an uncoupled condition, including a proximal mold portion 620 and a distal mold portion 640. It should be understood that the proximal mold portion 620 and distal mold portion 640 are shown in FIG. 14A in a cutaway view, and the mold portions may in practice have a shape represented by revolving the structures shown in FIG. 14A about a central longitudinal axis. In some examples, the blow mold 600 may be formed of a metal or metal alloy, although in other examples the blow mold 600 may be formed of plastic or another rigid material. In some examples, the proximal mold 620 and distal mold 640 may each include a relatively small end opening 622, 642, respectively. As should become clear, when the proximal mold 620 and distal mold 640 are assembled together, the relatively small end openings 622, 642 are positioned on opposite ends of the assembly. In some examples, the proximal mold 620 and distal mold 640 may each include a frustoconical or ramped section 624, 644 respectively, extending in a direction away from the relatively small end openings 622, 642. In some examples, the proximal mold 620 and distal mold 640 may each include a shoulder 626, 646 respectively, extending radially inward at a position extending in a direction away from the ramped sections 622, 644. In some examples, the proximal mold 620 and distal mold 640 may each include a central portion 628, 648 respectively, extending in a direction away from the shoulder 626, 646. Finally, in some examples, the proximal mold 620 and distal mold 640 may each include a connector 630, 650 respectively, at or near an end of the mold opposite the relatively small end openings 622, 624. In some examples, the shape of the proximal mold 620 may be substantially identical to the shape of the distal mold 640.

The proximal ramped section 624 and distal ramped section 644 may in some examples each end in a substantially straight segment having an inner diameter ID₂, ID₃, respectively that correspond to the outer diameters D₂, D₃ of balloon 536. In some examples, the interior surfaces of the proximal ramped section 624 and distal ramped section 644 have shapes and/or contours that are complementary to the shapes and/or contours of the exterior surfaces of proximal pillowed portion 536a and distal pillowed portion 536b of balloon 536. The central portions 628, 648 may in some examples each have an inner diameter ID₁ that corresponds to the outer diameter D₁ of balloon 536. In some examples, when the proximal mold 620 is assembled to the distal mold 640, the interior surfaces of assembled central portions 628, 648 have shapes and/or contours that are complementary to the shape and/or contour of the exterior surfaces of the crimp zone 536c of balloon 536. In some examples, the connectors 630, 650 are corresponding threads, although in other examples the connectors 630, 650 may be snap fit connectors, adhesives, or any other suitable connector. In some examples, in order to assemble the proximal mold 620 to the distal mold 640, they may be brought together in the direction of the arrows of FIG. 14A and then coupled using connectors 630, 650. In the assembled condition of the mold 600, as shown in FIG. 14B, in some examples it is preferably for the central portions 628, 648 to have a substantially smooth transition, although at least some minor seam may be expected. In the context of method 400 of FIG. 12B, the proximal mold 620 may be assembled to the distal mold 640 prior to, simultaneously with, or after step 402 and/or step 404.

Referring now in addition to FIG. 14B, FIG. 14B is a highly schematic view of the assembled mold 600 with an extruded tube 700 positioned therein. In the example of FIG. 14B, the extruded tube 700 has already been stretched or necked down to form the parison 720. In some examples, the extruded tube 700 may be formed similar to that described in connection with step 402 of method 400, and the parison 720 may in some examples be formed similar to that described in connection with step 404 or method 400. As explained above, the parison 720 may be formed as a generally uniform thickness structure, or with a thin-walled center section as described in connection with FIG. 12A. As should be clear from FIG. 14B, the tube 700 may pass thorough the relatively small end openings 622, 642 of mold portions 620, 640, respectively.

Referring now in addition to FIG. 14C, FIG. 14C is a highly schematic view of the parison 720 having been internally pressurized to blow the balloon 536 until the balloon 536 takes the general shape of the interior surface and/or contours of the assembled mold 600. In the example of FIG. 14C, spacing is intentionally shown between the balloon 536 and the mold 600 so that both structures are separately identifiable in the figure. However, in practical examples, after pressurizing the parison 720 to form the balloon 536, the balloon 536 may be in close contact with the inner surfaces of the assembled mold 600. In some examples, the configuration of FIG. 14C may correspond to third step 406 of method 400, after the balloon 536 has been blown but prior to optional additional processing such as pleating and/or folding. In some examples, heat is applied during the blowing of FIG. 14C, and in those examples the balloon 536 may be allowed to cool after the blowing. In some examples, after the balloon 536 has been blown, the balloon 536 will tend to revert to the blown shape when it is inflated in a future step.

Referring now in addition to FIG. 14D, FIG. 14D is a highly schematic example of the balloon mold 600 of FIG. 14C being disassembled after the balloon 536 has been formed. As shown in FIG. 14D, in some examples the proximal mold 620 may be disassembled from the distal mold 640, for example via unthreading or otherwise decoupling connector 630 from connector 650, and moving the mold portions 620, 640 in a direction away from each other represented by the arrows in FIG. 14D. In some examples, the tube 700 may be cut or otherwise removed just proximal to the proximal pillowed portion 536a and just distal to the distal pillowed portion 536b. In some examples, after forming the balloon 536 as shown in FIG. 14D, additional steps such as pleating and folding (e.g., step 408 of method 400) and pillowing (e.g., step 410 of method 400) may be performed on the balloon 536. In some examples, the balloon 536 may be assembled into a finished device similar to that described in connection with step 412 of method 400.

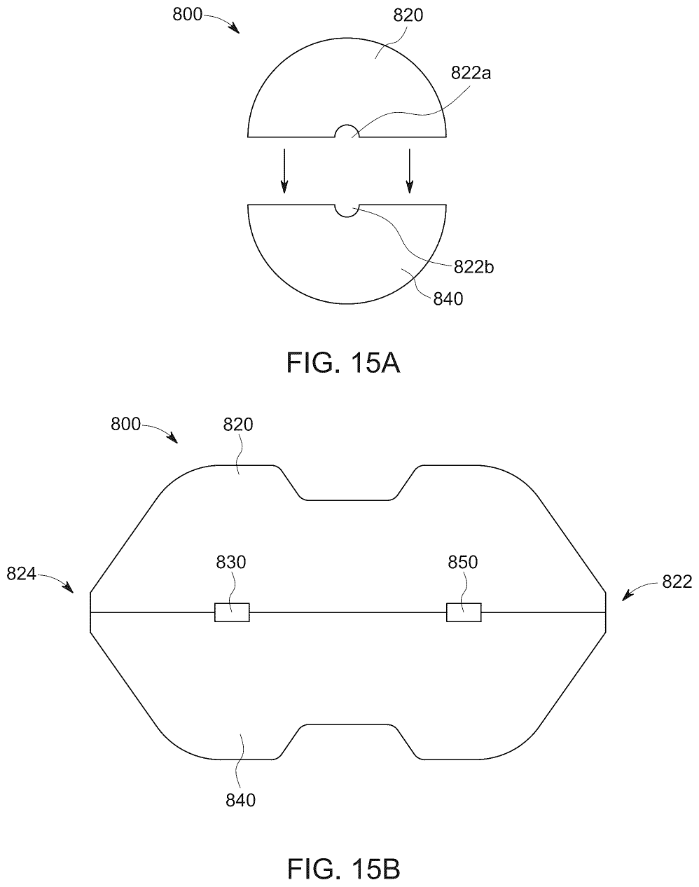

Referring now in addition to FIGS. 15A-15B, FIGS. 15A-15B are highly schematic views of another example of a balloon mold 800. Balloon mold 800 may include two portions that may be assembled together to have an assembled shape that is similar or identical to the assembled shape of balloon mold 600. In the end view of the example of FIG. 15A, balloon mold 800 includes a top mold portion 820 and a bottom mold portion 840. The top mold portion 820 and bottom mold portion may each define a portion (e.g., a half) of a proximal end opening 822 and a distal end opening 844. The two halves 822a, 822b of the proximal end opening 822 are visible in FIG. 15A, but the two halves of the distal end opening are not visible in the view of FIG. 15A. The two halves of the distal end opening would be visible and appear about identical to that shown in FIG. 15A in an end view taken on the opposite side of the mold 800 that is shown in FIG. 15A. When the top mold portion 820 is assembled to the bottom mold portion 840, it may form a shape that is substantially identical to the assembled mold 600 of FIG. 14B, and the use of the mold 800 may be substantially identical to the use of mold 600 except for the way in which the two mold portions are coupled (and decoupled). For example, FIG. 15B is a side view—not a cross-section—of the top mold 820 assembled to the bottom mold 840. Because FIG. 15B is a side view, the proximal end opening 822 and distal end opening 824 are not identifiable, but the positions of these openings are indicated in FIG. 15B. FIG. 15B illustrates examples of two couplers 830, 850 that may be used to couple the top mold portion 820 to the bottom mold portion 840. In some examples, the couplers 830, 850 may be latches. In other examples, they may be magnetic couplers, or other mechanical couplers besides latches. Although two individual couples 830, 850 are shown, more or fewer couplers may be provided, and the couplers may be provided on one or both sides of each portion 820, 840 of mold 800. In other examples, the top mold portion 820 and bottom mold portion 840 may be maintained together via hydraulic pressure from a clamping device.

Referring still to FIG. 15B, it should be understood that when the top mold portion 820 is assembled to the bottom mold portion 840, the interior shape of the assembled mold 800 may be substantially identical to that of mold 600, and the balloon 536 may be formed in substantially the same way using mold 800 as described in connection with using mold 600. The only difference of mold 800 is that it may be easier to decouple the top portion 820 from the bottom portion 840 after the balloon 546 is formed (similar to as shown in FIG. 14D, except the mold portions 820, 840 would separate vertically instead of horizontally). The only difference in the interior shape of the assembled mold 600 compared to the assembled mold 800 is the location and orientation of the seam where the two mold portions are coupled. For example, if balloon 536 is formed using mold 600, the resulting balloon 536 may include a circumferential seam within the crimp zone 536c where the two mold portions 620, 640 came together. However, if balloon 536 is formed using mold 800, the resulting balloon 536 may include two horizontal or axial seams on opposite sides of the balloon 536 where the two mold portions 820, 840 came together. Otherwise, the methodology described in connection with mold 600 applies fully to mold 800. In some examples, if a seam is formed in the balloon 536 due to the balloon 536 being molded within two separate mold portions, the seam may create a point of weakness in the balloon 536. In some examples, if balloon 536 were to burst during use, it may be preferable to control the location at which the burst occurs. In some examples, it may be desirable for a balloon to burst axially compared to radially, if a balloon were to burst. If mold 800 is used to form the balloon 536, it may allow for controlling the likely location of any burst to be along the axis of the balloon, and not along a radius of the balloon, which may be desirable. However, it is reiterated that other than the method of coupling and decoupling the mold portions, and the location of any potential resulting seam in the balloon 536, the shapes and uses of mold 600 and 800 may be identical, and thus the methodology of use of mold 800 is not repeated again here.

It should be understood that examples of balloon 536 are largely described in connection with creating a crimp zone 536c that creates a recessed area that is maintained during some or all of the inflation of balloon 536 to help maintain the axial position of the prosthetic heart valve 10 relative to the balloon 536 during valve deployment (and potentially during valve delivery as well), while examples of balloon 336 are largely described in connection with creating a thin-walled crimp zone 336c to help reduce the crimp profile of the system when the prosthetic heart valve 10 is crimped onto the balloon 336 for delivery. However, it should be understood that while these features may be implemented separately from each other, the may be implemented together in a single balloon.

It should be understood that the example method 200 described in connection with FIG. 11 (and variations thereof) may apply to the delivery and deployment of prosthetic heart valve 10 using delivery system 100 that includes balloon 336 (whether or not a reduced diameter crimp zone is provided with the thin-walled crimp zone 336c) or that includes balloon 536 (whether or not a thin-walled crimp zone is provided with the reduced diameter crimp zone 536c). Thus, the methods of use involving balloon 336 and/or balloon 536 may be similar to that described in connection with method 200, and thus the methods are not described again here. However, it should be noted that if a thin-walled crimp zone 336c is included, method steps 206 and 208 may in some examples be affected in the sense that the overall crimp profile during delivery and steering may be reduced. Also, it should be noted that if a reduced-diameter crimp zone 536c is included, method steps 212 and 216 may in some examples be affected because the prosthetic heart valve 10 may maintain a more stable axial position relative to the balloon 536 during balloon inflation. Otherwise, the methods of use involving balloons 336 and/or 536 may be substantially similar or identical to the methods of use involving balloon 136.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for delivering a prosthetic heart valve, the delivery system comprising:
a handle;
a delivery catheter extending from the handle; and
a balloon on a distal portion of the catheter, the balloon having a proximal portion, a distal portion, and a central portion positioned between the proximal portion and the distal portion, the central portion configured to receive the prosthetic heart valve in a crimped condition thereon,
wherein the proximal portion has a proximal wall thickness, the distal portion has a distal wall thickness, and the central portion has a central wall thickness, the central wall thickness being smaller than the proximal wall thickness and smaller than the distal wall thickness.

2. The system of claim 1, wherein the proximal wall thickness is about equal to the distal wall thickness.

3. The system of claim 1, wherein the proximal portion of the balloon, the distal portion of the balloon, and the central portion of the balloon are formed integrally with each other.

4. The system of claim 1, wherein the proximal portion of the balloon and the distal portion of the balloon each have a compliance that is greater than a compliance of the central portion of the balloon.

5. The system of claim 1, wherein the central wall thickness is between about 25% and about 75% of the proximal wall thickness.

6. The system of claim 5, wherein the central wall thickness is about 50% of the proximal wall thickness.

7. The system of claim 1, further comprising the prosthetic heart valve, wherein the prosthetic heart valve is a balloon-expandable prosthetic heart valve, the system having a delivery configuration in which the prosthetic heart valve is crimped so that it overlies the central portion of the balloon but does not overlie the proximal portion of the balloon or the distal portion of the balloon.

8. The system of claim 7, wherein in the delivery configuration of the system, the proximal portion of the balloon and the distal portion of the balloon each have an outer diameter that is greater or equal to an outer diameter of the crimped prosthetic heart valve.

9. The system of claim 1, wherein the balloon has a deflated delivery condition and an inflated deployment condition, an outer diameter of the distal portion of the balloon being greater than an outer diameter of the central portion of the balloon in the inflated deployment condition.

10. The system of claim 9, wherein an outer diameter of the proximal portion of the balloon is greater than the outer diameter of the central portion of the balloon in the inflated deployment condition.

11. A system for delivering a prosthetic heart valve, the delivery system comprising:
a handle;
a delivery catheter extending from the handle; and
a balloon on a distal portion of the catheter, the balloon having a proximal portion, a distal portion, and a central portion positioned between the proximal portion and the distal portion, the central portion configured to receive the prosthetic heart valve in a crimped condition thereon,
wherein when the balloon is in an inflated or blown state, the proximal portion has a proximal outer diameter, the distal portion has a distal outer diameter, and the central portion has a central outer diameter that is smaller than the proximal outer diameter and smaller than the distal outer diameter.

12. The system of claim 11, wherein the proximal portion of the balloon, the distal portion of the balloon, and the central portion of the balloon are formed integrally with each other.

13. The system of claim 11, further comprising the prosthetic heart valve, wherein the prosthetic heart valve is a balloon-expandable prosthetic heart valve, the system having a delivery configuration in which the prosthetic heart valve is crimped so that it overlies the central portion of the balloon but does not overlie the proximal portion of the balloon or the distal portion of the balloon.

14. The system of claim 13, wherein in an inflated condition of the balloon, a transition between the proximal portion of the balloon and the central portion of the balloon forms a proximal shoulder, and a transition between the distal portion of the balloon and the central portion of the balloon forms a distal shoulder.

15. The system of claim 14, wherein in the inflated condition of the balloon, an inflow end of the prosthetic heart valve abuts the distal shoulder and an outflow end of the prosthetic heart valve abuts the proximal shoulder.

16. The system of claim 11, wherein the proximal portion of the balloon has a proximal wall thickness, the distal portion of the balloon has a distal wall thickness, and the central portion of the balloon has a central wall thickness, the central wall thickness being smaller than the proximal wall thickness and smaller than the distal wall thickness.

17. The system of claim 16, wherein the proximal wall thickness is about equal to the distal wall thickness.

18. The system of claim 16, wherein the proximal portion of the balloon and the distal portion of the balloon each have a compliance that is greater than a compliance of the central portion of the balloon.

19. The system of claim 16, wherein the central wall thickness is between about 25% and about 75% of the proximal wall thickness.

20. The system of claim 19, wherein the central wall thickness is about 50% of the proximal wall thickness.
